(19) United States
(12) Patent Application Publication
(10) Pub. No.: US 2026/0327289 A1
(43) Pub. Date: Oct. 22, 2026

(54) METHODS OF IMPROVING LUNG DIFFUSION CAPACITY IN A PATIENT WITH PULMONARY ARTERIAL HYPERTENSION

(71) Applicant: Merck Sharp & Dohme LLC, Rahway, NJ (US)

(72) Inventor: Marius M. Hoeper, Hannover (DE)

(21) Appl. No.: 19/483,209

(22) PCT Filed: May 9, 2024
(86) PCT No.: PCT/US2024/028555

§ 371 Date: Nov. 10, 2025

Related U.S. Application Data
(60) Provisional application No. 63/466,014, filed on May 12, 2023.

(51) Int. Cl. C07K 14/71; A61K 38/00; A61K 47/68; A61P 9/12; A61P 11/00

(52) U.S. Cl. CPC C07K 14/71; A61K 47/6811; A61P 9/12; A61P 11/00; A61K 38/00; C07K 2319/30

(57) ABSTRACT

This disclosure relates generally to methods of improving lung diffusion capacity in a patient with pulmonary arterial hypertension comprising administering an ActRIIA fusion protein, such as sotatercept to the patient.

### CROSS-REFERENCE TO RELATED APPLICATIONS

This application claims the benefit of priority from U.S. Provisional Application No. 63/466,014, filed May 12, 2023. The foregoing application is incorporated herein by reference.

### REFERENCE TO SEQUENCE LISTING SUBMITTED ELECTRONICALLY

The sequence listing of the present application is submitted electronically as an XML formatted sequence listing created on Apr. 17, 2024, with the file name 1848179-0002-171-WO1_Sequence_Listing and having a size of 21.4 kb. This sequence listing submitted electronically is part of the specification and is herein incorporated by reference in its entirety.

### FIELD

This disclosure relates generally to methods of improving lung diffusion capacity for carbon monoxide and in gas exchange in a patient with pulmonary arterial hypertension comprising administering an ActRIIA fusion protein to the patient. In some embodiments, the ActRIIA fusion protein is sotatercept.

### BACKGROUND

Pulmonary arterial hypertension (PAH) is a rare disease characterized by remodeling of small pulmonary vessels, which results in a progressive increase in right ventricular afterload. Most of the symptoms and signs of PAH are attributable to impaired right heart function, and the disease continues to have a high mortality, with right heart failure being the leading cause of death in this patient population. However, in addition to haemodynamic impairment, patients with PAH show variable alterations in their lung diffusion capacity for carbon monoxide (DLCO) and in gas exchange which contribute to disease symptoms and severity.

Over the past two decades, a variety of drugs have been approved for the treatment of PAH, and several new compounds are currently being explored. The clinical trials that have investigated the safety and efficacy of these medications focused on haemodynamics, exercise capacity, and clinical outcomes, while the effects on DLCO and gas exchange were not studied. Retrospective analyses of registry data suggested that there is usually little change in the DLCO with initiation of PAH therapies whereas the effects on oxygenation are variable, with some patients showing deteriorations.

A compound currently under investigation for therapeutic use in PAH is sotatercept, a fusion protein consisting of human activin receptor type IIA linked to the extracellular Fc (fragment, crystallizable) domain of human immunoglobulin G. Sotatercept acts as a ligand trap for select transforming growth factor-β superfamily members including activin A, activin B, and growth differentiation factors 8 and 11. Inactivation of these ligands by sotatercept is thought to rebalance pulmonary vascular homeostasis toward growth inhibiting and pro-apoptotic signaling. Sotatercept has shown efficacy in PULSAR, a phase 2 trial and STELLAR, a phase 3 trial, both randomized, double-blind, placebo-controlled studies, which together have demonstrated improvements in haemodynamics, exercise capacity, right heart function, time to clinical worsening, and quality of life in patients with PAH.

Furthermore, it is desirable for a treatment for PAH, such as sotatercept to improve lung diffusion capacity for carbon monoxide and gas exchange in patients with PAH.

### SUMMARY

The disclosure provides a method of improving lung diffusion capacity in a patient with pulmonary arterial hypertension comprising administering an ActRIIA fusion protein to the patient. For example, the disclosure provides a method of improving lung diffusion capacity in a patient with pulmonary arterial hypertension comprising administering an ActRIIA fusion protein to the patient, wherein lung diffusion capacity in the patient is improved following administration of sotatercept.

The disclosure provides a method of improving lung diffusion capacity in a patient with pulmonary arterial hypertension comprising administering sotatercept to the patient. The disclosure also provides a method of improving lung diffusion capacity in a patient with pulmonary arterial hypertension comprising administering sotatercept to the patient, wherein lung diffusion capacity in the patient is improved following administration of sotatercept.

The disclosure provides a method of improving lung diffusion capacity in a patient with pulmonary arterial hypertension comprising administering an ActRIIA fusion protein to the patient, wherein the ActRIIA fusion protein has the amino acid sequence of SEQ ID NO: 4.

The disclosure provides a method of improving lung diffusion capacity in a patient with pulmonary arterial hypertension comprising administering an ActRIIA fusion protein to the patient, wherein the ActRIIA fusion protein has the amino acid sequence of SEQ ID NO: 5.

The disclosure provides a method of improving lung diffusion capacity in a patient with pulmonary arterial hypertension comprising administering an ActRIIA fusion protein to the patient, wherein the ActRIIA fusion protein comprises SEQ ID NO: 1 linked to a Fc region by a linker.

In certain embodiments, the lung diffusion capacity is lung diffusion capacity for carbon monoxide. In certain embodiments, the diffusion capacity for carbon monoxide in a patient with pulmonary arterial hypertension is improved. In certain embodiments, the diffusion capacity for carbon monoxide in a patient with pulmonary arterial hypertension is increased.

In certain embodiments, the diffusion capacity for carbon monoxide in a patient with pulmonary arterial hypertension is increased by at least 4%. In certain embodiments, the diffusion capacity for carbon monoxide in a patient with pulmonary arterial hypertension is increased by at least 5%. In certain embodiments, the diffusion capacity for carbon monoxide in a patient with pulmonary arterial hypertension is increased by at least 6%. In certain embodiments, the diffusion capacity for carbon monoxide in a patient with pulmonary arterial hypertension is increased by at least 7%. In certain embodiments, the diffusion capacity for carbon monoxide in a patient with pulmonary arterial hypertension is increased by at least 8%.

In certain embodiments, the diffusion capacity for carbon monoxide in a patient with pulmonary arterial hypertension is increased by 4%. In certain embodiments, the diffusion capacity for carbon monoxide in a patient with pulmonary arterial hypertension is increased by 5%. In certain embodiments, the diffusion capacity for carbon monoxide in a patient with pulmonary arterial hypertension is increased by 6%. In certain embodiments, the diffusion capacity for carbon monoxide in a patient with pulmonary arterial hypertension is increased by 7%. In certain embodiments, the diffusion capacity for carbon monoxide in a patient with pulmonary arterial hypertension is increased by 8%.

The disclosure also provides a method of improving gas exchange in a patient with pulmonary arterial hypertension comprising administering an ActRIIA fusion protein to the patient. The disclosure also provides a method of improving gas exchange in a patient with pulmonary arterial hypertension comprising administering an ActRIIA fusion protein to the patient, wherein gas exchange is improved in the patient following administration of sotatercept.

The disclosure also provides a method of improving gas exchange in a patient with pulmonary arterial hypertension comprising administering sotatercept to the patient. The disclosure also provides a method of improving gas exchange in a patient with pulmonary arterial hypertension comprising administering sotatercept to the patient, wherein gas exchange is improved in the patient following administration of sotatercept.

The disclosure also provides a method of improving gas exchange in a patient with pulmonary arterial hypertension comprising administering an ActRIIA fusion protein to the patient, wherein the ActRIIA fusion protein has the amino acid sequence of SEQ ID NO: 4.

The disclosure also provides a method of improving gas exchange in a patient with pulmonary arterial hypertension comprising administering an ActRIIA fusion protein to the patient, wherein the ActRIIA fusion protein has the amino acid sequence of SEQ ID NO: 5.

The disclosure also provides a method of improving gas exchange in a patient with pulmonary arterial hypertension comprising administering an ActRIIA fusion protein to the patient, wherein the ActRIIA fusion protein comprises the amino acid sequence of SEQ ID NO: 1, linked to a Fc portion via a linker.

The disclosure also provides a method of improving oxygenation in a patient with pulmonary arterial hypertension comprising administering an ActRIIA fusion protein to the patient. The disclosure also provides a method of improving oxygenation in a patient with pulmonary arterial hypertension comprising administering an ActRIIA fusion protein to the patient, wherein oxygenation in the patient is improved following administration of sotatercept.

The disclosure also provides a method of improving oxygenation in a patient with pulmonary arterial hypertension comprising administering sotatercept to the patient. The present disclosure also provides a method of improving oxygenation in a patient with pulmonary arterial hypertension comprising administering sotatercept to the patient, wherein oxygenation in the patient is improved following administration of sotatercept.

The disclosure also provides a method of improving oxygenation in a patient with pulmonary arterial hypertension comprising administering an ActRIIA fusion protein to the patient, wherein the ActRIIA fusion protein has the amino acid sequence of SEQ ID NO: 4.

The disclosure also provides a method of improving oxygenation in a patient with pulmonary arterial hypertension comprising administering an ActRIIA fusion protein to the patient, wherein the ActRIIA fusion protein has the amino acid sequence of SEQ ID NO: 5.

The disclosure also provides a method of improving oxygenation in a patient with pulmonary arterial hypertension comprising administering an ActRIIA fusion protein to the patient, wherein the ActRIIA fusion protein comprises the amino acid sequence of SEQ ID NO: 1, linked to a Fc portion via a linker.

In certain embodiments, the oxygenation is arterial oxygen tension [PaO₂]. In certain embodiments, the arterial oxygen tension is increased. In certain embodiments, the arterial oxygen tension is increased by at least 5 mmHg. In certain embodiments, the arterial oxygen tension is increased by at least 6 mmHg. In certain embodiments, the arterial oxygen tension is increased by at least 7 mmHg. In certain embodiments, the arterial oxygen tension is increased by at least 8 mmHg. In certain embodiments, the arterial oxygen tension is increased by at least 9 mmHg. In certain embodiments, the arterial oxygen tension is increased by at least 10 mmHg.

In certain embodiments, the arterial oxygen tension is increased by 5 mmHg. In certain embodiments, the arterial oxygen tension is increased by 6 mmHg. In certain embodiments, the arterial oxygen tension is increased by 7 mmHg. In certain embodiments, the arterial oxygen tension is increased by 8 mmHg. In certain embodiments, the arterial oxygen tension is increased by 9 mmHg. In certain embodiments, the arterial oxygen tension is increased by 10 mmHg.

In certain embodiments of the methods described herein, sotatercept is administered to the patient at a dose from 0.3 mg/kg of body weight of the patient to 0.7 mg/kg. In certain embodiments of the methods described herein, sotatercept is administered to the patient at a dose of 0.3 mg/kg. In certain embodiments of the methods described herein, sotatercept is administered to the patient at a dose of 0.4 mg/kg. In certain embodiments of the methods described herein, sotatercept is administered to the patient at a dose of 0.5 mg/kg. In certain embodiments of the methods described herein, sotatercept is administered to the patient at a dose of 0.6 mg/kg. In certain embodiments of the methods described herein, sotatercept is administered to the patient at a dose of 0.7 mg/kg.

In certain embodiments of the methods described herein, sotatercept is administered about once every three weeks. In certain embodiments of the methods described herein, sotatercept is administered once every three weeks.

In certain embodiments of the methods described herein, sotatercept is administered intravenously. In certain embodiments of the methods described herein, sotatercept is administered subcutaneously.

The summary of the technology described above is non-limiting and other features and advantages of the technology will be apparent from the following detailed description, and from the claims.

### BRIEF DESCRIPTION OF THE DRAWINGS

FIG. 1 shows violine plots showing density distribution and individual measurements of DLCO, PaO₂, PaCO₂ and SaO₂ at baseline and week 24 in study participants receiving placebo or sotatercept.

FIG. 2. shows violine plots showing density distribution and individual measurements of DLCO, PaO₂, PaCO₂ and SaO₂ at baseline and week 24 in patients who were originally assigned to the placebo arm of their respective studies who showed improvements in DLCO and PaO₂ after initiation of sotatercept therapy.

### DETAILED DESCRIPTION

### Definitions and Abbreviations

### Abbreviations

BMI body mass index;
CHD congenital heart disease;
WHO FC World Health Organization functional class
NT-proBNP N-terminal fragment of pro-brain natriuretic peptide
TLC total lung capacity
FVC forced vital capacity
FEV1 forced expiratory volume in 1 second
DLCO lung diffusion capacity for carbon monoxide
PaO₂ arterial oxygen tension
PaCO₂ arterial carbon dioxide tension
SaO₂ arterial oxygen saturation
RAP right atrial pressure
mPAP mean pulmonary arterial pressure
PAWP pulmonary artery wedge pressure
PVR pulmonary vascular resistance
SvO₂ mixed venous oxygen saturation
SC subcutaneous
IV intravenous
CI confidence interval

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

“Administration” and “treatment,” as it applies to an animal, human, subject, cell, tissue, organ, or biological fluid, refers to contact of an exogenous pharmaceutical, therapeutic, diagnostic agent, or composition to the animal, human, subject, cell, tissue, organ, or biological fluid. “Treat” or “treating” as used herein, means to administer an ActRIIA fusion protein, to a subject having PAH, suspected of having PAH, or diagnosed with PAH, to achieve at least one positive therapeutic effect, such as for example, improvement in improving lung diffusion capacity for carbon monoxide and in gas exchange. “Treatment” may include one or more of the following: increasing lung diffusion capacity for carbon monoxide or an increasing gas exchange.

The term “patient” (alternatively referred to as “subject” or “individual” herein) refers to a mammal (e.g., rat, mouse, dog, cat, rabbit) capable of being treated with the methods and compositions of the invention, most preferably a human. In some embodiments, the patient is an adult patient. In other embodiments, the patient is a pediatric patient.

The term “ActRIIA” (alternatively ActRIIa) includes polypeptides comprising any naturally occurring polypeptide of an ActRIIA family member as well as any variants thereof (including mutants, fragments, fusions, and peptidomimetic forms) that retain a useful activity.

The term “ActRIIA fusion proteins” includes an ActRIIA polypeptide fused to an Fc region via a linker.

The term “oxygenation” means providing greater oxygen supply to the lungs.

### ActRIIA Fusion Proteins

The canonical human ActRIIA precursor protein sequence is as follows:

| (SEQ ID NO: 12) | |
|---|---|
| 1 | MGAAAKLAFA VFLISCSSGA ILGRSETQEC LFFNANWEKD RT [symbol] QTGVEPC |
| 51 | YGDKDKRRHC FATWK [symbol] ISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 201 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLINIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A processed (mature) extracellular human ActRIIA polypeptide sequence is as follows:

(SEQ ID NO: 1)
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPP

The C-terminal “tail” of the extracellular domain is indicated by single underline. The sequence with the “tail” deleted (a Δ15 sequence) is as follows:

(SEQ ID NO: 13)
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EM

A nucleic acid sequence encoding the human ActRIIA precursor protein (SEQ ID NO: 12) is shown below (SEQ ID NO: 14), as follows nucleotides 159-1700 of Genbank Reference Sequence NM_001616.4. The signal sequence is underlined.

| (SEQ ID NO: 14) | |
|---|---|
| 1 | atgggagctg ctgcaaagtt ggcgtttgcc gtctttctta tctcctgttc |
| 51 | ttcaggtgct atacttggta gatcagaaac tcaggagtgt cttttcttta |
| 101 | atgctaattg ggaaaaagac agaaccaatc aaactggtgt tgaaccgtgt |
| 151 | tatggtgaca aagataaacg gcggcattgt tttgctacct ggaagaatat |
| 201 | ttctggttcc attgaaatag tgaaacaagg ttgttggctg gatgatatca |
| 251 | actgctatga caggactgat tgtgtagaaa aaaaagacag ccctgaagta |
| 301 | tatttttgtt gctgtgaggg caatatgtgt aatgaaaagt tttcttattt |
| 351 | tccggagatg gaagtcacac agcccacttc aaatccagtt acacctaagc |
| 401 | caccctatta caacatcctg ctctattcct tggtgccact tatgttaatt |
| 451 | gcggggattg tcatttgtgc attttgggtg tacaggcatc acaagatggc |
| 501 | ctaccctcct gtacttgttc caactcaaga cccaggacca cccccacctt |
| 551 | ctccattact aggtttgaaa ccactgcagt tattagaagt gaaagcaagg |
| 601 | ggaagatttg gttgtgtctg gaaagcccag ttgcttaacg aatatgtggc |
| 651 | tgtcaaaata tttccaatac aggacaaaca gtcatggcaa aatgaatacg |
| 701 | aagtctacag tttgcctgga atgaagcatg agaacatatt acagttcatt |
| 751 | ggtgcagaaa aacgaggcac cagtgttgat gtggatcttt ggctgatcac |
| 801 | agcatttcat gaaaagggtt cactatcaga ctttcttaag gctaatgtgg |
| 851 | tctcttggaa tgaactgtgt catattgcag aaaccatggc tagaggattg |
| 901 | gcatatttac atgaggatat acctggccta aaagatggcc acaaacctgc |
| 951 | catatctcac agggacatca aaagtaaaaa tgtgctgttg aaaaacaacc |
| 1001 | tgacagcttg cattgctgac tttgggttgg ccttaaaatt tgaggctggc |
| 1051 | aagtctgcag gcgataccca tggacaggtt ggtacccgga ggtacatggc |
| 1101 | tccagaggta ttagagggtg ctataaactt ccaaagggat gcatttttga |
| 1151 | ggatagatat gtatgccatg ggattagtcc tatgggaact ggcttctcgc |
| 1201 | tgtactgctg cagatggacc tgtagatgaa tacatgttgc catttgagga |
| 1251 | ggaaattggc cagcatccat ctcttgaaga catgcaggaa gttgttgtgc |
| 1301 | ataaaaaaaa gaggcctgtt ttaagagatt attggcagaa acatgctgga |
| 1351 | atggcaatgc tctgtgaaac cattgaagaa tgttgggatc acgacgcaga |
| 1401 | agccaggtta tcagctggat gtgtaggtga aagaattacc cagatgcaga |
| 1451 | gactaacaaa tattattacc acagaggaca ttgtaacagt ggtcacaatg |
| 1501 | gtgacaaatg ttgactttcc toccaaagaa tctagtcta |

A nucleic acid sequence encoding the processed soluble (extracellular) human ActRIIA polypeptide (SEQ ID NO: 1) is as follows:

| (SEQ ID NO: 15) | |
|---|---|
| 1 | atacttggta gatcagaaac tcaggagtgt cttttcttta atgctaattg |
| 51 | ggaaaaagac agaaccaatc aaactggtgt tgaaccgtgt tatggtgaca |
| 101 | aagataaacg gcggcattgt tttgctacct ggaagaatat ttctggttcc |
| 151 | attgaaatag tgaaacaagg ttgttggctg gatgatatca actgctatga |
| 201 | caggactgat tgtgtagaaa aaaaagacag ccctgaagta tatttttgtt |
| 251 | gctgtgaggg caatatgtgt aatgaaaagt tttcttattt tccggagatg |
| 301 | gaagtcacac agcccacttc aaatccagtt acacctaagc caccc |

In some embodiments, the ActRIIA polypeptide sequence comprises the sequence disclosed at accession number UniProtKB/Swiss-Prot P27037.1, and/or variants thereof. In some embodiments, the term “wild-type ActRIIA polypeptide” refers to the extracellular domain of ActRIIA, amino acids 1 to 135 (with signal sequence), or amino acids 20 through 135 of SEQ ID NO: 1 (without signal sequence)

Sotatercept is a recombinant fusion protein consisting of the wild-type extracellular domain (ECD) of human activin receptor type IIA (SEQ ID NO: 1) (ActRIIA, encoded by ACVR2A) fused by a short linker TGGG (SEQ ID NO: 2) to a human IgG1 Fc domain,

(SEQ ID NO: 3)
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPVPIEKTISKAKGQPREPQVYTLPPSREEMTKNQVSLTCLVKGF
YPSDIAVEWESNGQPENNYKTTPPVLDSDGSFFLYSKLTVDKSRWQQGNV
FSCSVMHEALHNHYTQKSLSLSPGK.

The full sequence of sotatercept (SEQ ID NO:4) is as follows:

(SEQ ID NO: 4)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPPTGGGTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK.

Sotatercept can be susceptible to post translational modification including C-terminal lysine clipping. Sotatercept lacking the C-terminal lysine is shown below as purified from CHO cell lines (SEQ ID NO: 5):

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPPTGGGTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPG

### Methods and Uses of the Invention

The invention is directed to a method of improving lung diffusion capacity in a patient with pulmonary arterial hypertension comprising administering sotatercept to the patient, wherein lung diffusion capacity is improved in the patient following administration of sotatercept.

The disclosure also provides a method of improving gas exchange in a patient with pulmonary arterial hypertension comprising administering sotatercept to the patient, wherein gas exchange is improved in the patient following administration of sotatercept.

The disclosure also provides a method of improving oxygenation in a patient with pulmonary arterial hypertension comprising administering sotatercept to the patient, wherein oxygenation is improved in the patient following administration of sotatercept.

In certain embodiments, the invention is directed to a method of improving lung diffusion capacity in a patient with pulmonary arterial hypertension comprising administering an ActRIIA fusion protein to a patient, wherein the ActRIIA fusion protein comprises the amino acid sequence of SEQ ID NO: 1; an Fc domain of an immunoglobulin; and a linker domain positioned between SEQ ID NO: 1 and the Fc domain, wherein lung diffusion capacity is improved in the patient following administration of the fusion protein. In certain embodiments, the invention is directed to a method of improving lung diffusion capacity in a patient with pulmonary arterial hypertension comprising administering an ActRIIA fusion protein to a patient, wherein the ActRIIA fusion protein comprises an amino acid sequence that is at least 90% identical to the amino acid sequence of SEQ ID NO: 1; an Fc domain of an immunoglobulin; and a linker domain positioned between SEQ ID NO: 1 and the Fc domain. In certain embodiments, invention is directed to a method of improving lung diffusion capacity in a patient with pulmonary arterial hypertension comprising administering an ActRIIA fusion protein to a patient, wherein the ActRIIA fusion protein comprises an amino acid sequence that is at least 95% identical to the amino acid sequence of SEQ ID NO: 1; an Fc domain of an immunoglobulin; and a linker domain positioned between SEQ ID NO: 1 and the Fc domain. In certain embodiments, the invention is directed to a method of improving lung diffusion capacity in a patient with pulmonary arterial hypertension comprising administering an ActRIIA fusion protein to a patient, wherein the ActRIIA fusion protein comprises an amino acid sequence that is at least 97% identical to the amino acid sequence of SEQ ID NO: 1; an Fc domain of an immunoglobulin; and a linker domain positioned between SEQ ID NO: 1 and the Fc domain. In certain embodiments, the invention is directed to a method of improving lung diffusion capacity in a patient with pulmonary arterial hypertension comprising administering an ActRIIA fusion protein to a patient, wherein the ActRIIA fusion protein comprises an amino acid sequence that is at least 99% identical to the amino acid sequence of SEQ ID NO: 1; an Fc domain of an immunoglobulin; and a linker domain positioned between SEQ ID NO: 1 and the Fc domain.

The disclosure also provides a method of improving gas exchange in a patient with pulmonary arterial hypertension comprising administering an ActRIIA fusion protein to a patient, wherein the ActRIIA fusion protein comprises the amino acid sequence of SEQ ID NO: 1; an Fc domain of an immunoglobulin; and a linker domain positioned between SEQ ID NO: 1 and the Fc domain, wherein gas exchange is improved in the patient following administration of the fusion protein. The disclosure also provides a method of improving gas exchange in a patient with pulmonary arterial hypertension comprising administering an ActRIIA fusion protein to a patient, wherein the ActRIIA fusion protein comprises an amino acid sequence that is at least 90% identical to the amino acid sequence of SEQ ID NO: 1; an Fc domain of an immunoglobulin; and a linker domain positioned between SEQ ID NO: 1 and the Fc domain. The disclosure also provides a method of improving gas exchange in a patient with pulmonary arterial hypertension comprising administering an ActRIIA fusion protein to a patient, wherein the ActRIIA fusion protein comprises an amino acid sequence that is at least 95% identical to the amino acid sequence of SEQ ID NO: 1; an Fc domain of an immunoglobulin; and a linker domain positioned between SEQ ID NO: 1 and the Fc domain. The disclosure also provides a method of improving gas exchange in a patient with pulmonary arterial hypertension comprising administering an ActRIIA fusion protein to a patient, wherein the ActRIIA fusion protein comprises an amino acid sequence that is at least 97% identical to the amino acid sequence of SEQ ID NO: 1; an Fc domain of an immunoglobulin; and a linker domain positioned between SEQ ID NO: 1 and the Fc domain. The disclosure also provides a method of improving gas exchange in a patient with pulmonary arterial hypertension comprising administering an ActRIIA fusion protein to a patient, wherein the ActRIIA fusion protein comprises an amino acid sequence that is at least 99% identical to the amino acid sequence of SEQ ID NO: 1; an Fc domain of an immunoglobulin; and a linker domain positioned between SEQ ID NO: 1 and the Fc domain.

The disclosure also provides a method of improving oxygenation in a patient with pulmonary arterial hypertension comprising administering an ActRIIA fusion protein to a patient, wherein the ActRIIA fusion protein comprises the amino acid sequence of SEQ ID NO: 1; an Fc domain of an immunoglobulin; and a linker domain positioned between SEQ ID NO: 1 and the Fc domain, wherein oxygenation is improved in the patient following administration of the fusion protein. The disclosure also provides a method of improving oxygenation in a patient with pulmonary arterial hypertension comprising administering an ActRIIA fusion protein to a patient, wherein the ActRIIA fusion protein comprises an amino acid sequence that is at least 90% identical to the amino acid sequence of SEQ ID NO: 1; an Fc domain of an immunoglobulin; and a linker domain positioned between SEQ ID NO: 1 and the Fc domain. The disclosure also provides a method of improving oxygenation in a patient with pulmonary arterial hypertension comprising administering an ActRIIA fusion protein to a patient, wherein the ActRIIA fusion protein comprises an amino acid sequence that is at least 95% identical to the amino acid sequence of SEQ ID NO: 1; an Fc domain of an immunoglobulin; and a linker domain positioned between SEQ ID NO: 1 and the Fc domain. The disclosure also provides a method of improving oxygenation in a patient with pulmonary arterial hypertension comprising administering an ActRIIA fusion protein to a patient, wherein the ActRIIA fusion protein comprises an amino acid sequence of that is at least 97% identical to the amino acid sequence SEQ ID NO: 1; an Fc domain of an immunoglobulin; and a linker domain positioned between SEQ ID NO: 1 and the Fc domain. The disclosure also provides a method of improving oxygenation in a patient with pulmonary arterial hypertension comprising administering an ActRIIA fusion protein to a patient, wherein the ActRIIA fusion protein comprises an amino acid sequence of that is at least 99% identical to the amino acid sequence SEQ ID NO: 1; an Fc domain of an immunoglobulin; and a linker domain positioned between SEQ ID NO: 1 and the Fc domain.

In certain embodiments, the linker domain is selected from the group consisting of:

(SEQ ID NO: 2)
TGGG,
(SEQ ID NO: 6)
TGGGG,
(SEQ ID NO: 7)
SGGGG,
(SEQ ID NO: 8)
GGGGS,
(SEQ ID NO: 9)
GGG,
(SEQ ID NO: 10)
GGGG,
and
(SEQ ID NO: 11)
SGGG.

In certain embodiments, the linker domain is TGGG (SEQ ID NO: 2).

In certain embodiments, the Fc domain comprises the amino acid sequence of SEQ ID NO: 3. In certain embodiments, the Fc domain consists of the amino acid sequence of SEQ ID NO: 3. In certain embodiments, the Fc domain is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO: 3.

In certain embodiments, the invention is directed to a method of improving lung diffusion capacity in a patient with pulmonary arterial hypertension comprising administering an ActRIIA fusion protein to a patient, wherein the ActRIIA fusion protein comprises the amino acid sequence of SEQ ID NO: 4, wherein lung diffusion capacity is improved in the patient following administration of the fusion protein. In certain embodiments, the invention is directed to a method of improving lung diffusion capacity in a patient with pulmonary arterial hypertension comprising administering an ActRIIA fusion protein that is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO: 4, to the patient.

The disclosure also provides a method of improving gas exchange in a patient with pulmonary arterial hypertension comprising administering an ActRIIA fusion protein to a patient, wherein the ActRIIA fusion protein comprises the amino acid sequence of SEQ ID NO: 4, wherein gas exchange is improved in the patient following administration of the fusion protein. The disclosure also provides a method of improving gas exchange in a patient with pulmonary arterial hypertension comprising administering an ActRIIA fusion protein that is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO: 4 to the patient.

The disclosure also provides a method of improving oxygenation in a patient with pulmonary arterial hypertension comprising administering an ActRIIA fusion protein to a patient, wherein the ActRIIA fusion protein comprises the amino acid sequence of SEQ ID NO: 4, wherein oxygenation is improved in the patient following administration of the fusion protein. The disclosure also provides a method of improving oxygenation in a patient with pulmonary arterial hypertension comprising administering an ActRIIA fusion protein that is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO: 4 to a patient.

In certain embodiments, the invention is directed to a method of improving lung diffusion capacity in a patient with pulmonary arterial hypertension comprising administering an ActRIIA fusion protein to a patient, wherein the ActRIIA fusion protein comprises the amino acid sequence of SEQ ID NO: 5. In certain embodiments, the invention is directed to a method of improving lung diffusion capacity in a patient with pulmonary arterial hypertension comprising administering an ActRIIA fusion protein that is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO: 5 to the patient.

The disclosure also provides a method of improving gas exchange in a patient with pulmonary arterial hypertension comprising administering an ActRIIA fusion protein to a patient, wherein the ActRIIA fusion protein comprises the amino acid sequence of SEQ ID NO: 5, wherein gas exchange is improved in the patient following administration of the fusion protein. The disclosure also provides a method of improving gas exchange in a patient with pulmonary arterial hypertension comprising administering an ActRIIA fusion protein that is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO: 5 to the patient.

The disclosure also provides a method of improving oxygenation in a patient with pulmonary arterial hypertension comprising administering an ActRIIA fusion protein to a patient, wherein the ActRIIA fusion protein comprises the amino acid sequence of SEQ ID NO: 5, wherein oxygenation is improved in the patient following administration of the fusion protein. The disclosure also provides a method of improving oxygenation in a patient with pulmonary arterial hypertension comprising administering an ActRIIA fusion protein that is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO: 5 to the patient.

In certain embodiments, the lung diffusion capacity is lung diffusion capacity for carbon monoxide (DLCO). In certain embodiments, the DLCO in a patient with pulmonary arterial hypertension is increased. In certain embodiments, the DLCO in a patient with pulmonary arterial hypertension is increased by at least 4%. In certain embodiments, the DLCO in a patient with pulmonary arterial hypertension is increased by at least 5%. In certain embodiments, the DLCO in a patient with pulmonary arterial hypertension is increased by at least 6%. In certain embodiments, the DLCO in a patient with pulmonary arterial hypertension is increased by at least 7%. In certain embodiments, the DLCO in a patient with pulmonary arterial hypertension is increased by at least 8%.

In certain embodiments, the diffusion capacity for carbon monoxide (DLCO) in a patient with pulmonary arterial hypertension is increased by 4%. In certain embodiments, the DLCO in a patient with pulmonary arterial hypertension is increased by 5%. In certain embodiments, the DLCO in a patient with pulmonary arterial hypertension is increased by 6%. In certain embodiments, the DLCO in a patient with pulmonary arterial hypertension is increased by 7%. In certain embodiments, the DLCO in a patient with pulmonary arterial hypertension is increased by 8%.

In certain embodiments, the oxygenation is measured by arterial oxygen tension [PaO₂], which measures the partial pressure of gas (oxygen or carbon dioxide) in the blood. In certain embodiments, the arterial oxygen tension is increased. In certain embodiments, the arterial oxygen tension is increased by at least 5 mmHg. In certain embodiments, the arterial oxygen tension is increased by at least 6 mmHg. In certain embodiments, the arterial oxygen tension is increased by at least 7 mmHg. In certain embodiments, the arterial oxygen tension is increased by at least 8 mmHg. In certain embodiments, the arterial oxygen tension is increased by at least 9 mmHg. In certain embodiments, the arterial oxygen tension is increased by at least 10 mmHg.

In certain embodiments, the arterial oxygen tension is increased by 5 mmHg. In certain embodiments, the arterial oxygen tension is increased by 6 mmHg. In certain embodiments, the arterial oxygen tension is increased by 7 mmHg. In certain embodiments, the arterial oxygen tension is increased by 8 mmHg. In certain embodiments, the arterial oxygen tension is increased by 9 mmHg. In certain embodiments, the arterial oxygen tension is increased by 10 mmHg.

In certain embodiments, the oxygenation is measured by mixed venous oxygen saturation [SvO₂]. In certain embodiments, the mixed venous oxygen saturation in a patient with pulmonary arterial hypertension is increased. In certain embodiments, the mixed venous oxygen saturation in a patient with pulmonary arterial hypertension is increased by at least 4%. In certain embodiments, the mixed venous oxygen saturation in a patient with pulmonary arterial hypertension is increased by at least 5%. In certain embodiments, the mixed venous oxygen saturation in a patient with pulmonary arterial hypertension is increased by at least 6%. In certain embodiments, the mixed venous oxygen saturation in a patient with pulmonary arterial hypertension is increased by at least 7%. In certain embodiments, the mixed venous oxygen saturation in a patient with pulmonary arterial hypertension is increased by at least 8%.

In certain embodiments, the mixed venous oxygen saturation in a patient with pulmonary arterial hypertension is increased by 4%. In certain embodiments, the mixed venous oxygen saturation in a patient with pulmonary arterial hypertension is increased by 5%. In certain embodiments, the mixed venous oxygen saturation in a patient with pulmonary arterial hypertension is increased by 6%. In certain embodiments, the mixed venous oxygen saturation in a patient with pulmonary arterial hypertension is increased by 7%. In certain embodiments, the mixed venous oxygen saturation in a patient with pulmonary arterial hypertension is increased by 8%.

In certain embodiments, the therapeutic methods of the invention include administering the ActRIIA fusion proteins systemically or locally.

Typically, protein therapeutic agents disclosed herein will be administered parentally, and particularly intravenously or subcutaneously.

In some embodiments, the methods of the invention comprise administering an ActRIIA fusion protein using a dosing regimen. In some embodiments, the method comprises administering a therapeutically effective amount of an ActRIIA fusion protein as disclosed herein to a patient, comprising a first dose of between 0.1 mg/kg and 1.0 mg/kg of the ActRIIA fusion protein for a first period of time, and a second dose of between 0.1 mg/kg and 1.0 mg/kg of the ActRIIA fusion protein subsequently administered for a second period of time. In some embodiments, the method comprises administering a therapeutically effective amount of an ActRIIA fusion protein as disclosed herein to a patient, comprising a first dose of between 0.1 mg/kg and 1.0 mg/kg of ActRIIA fusion protein for a first period of time, a second dose of between 0.1 mg/kg and 1.0 mg/kg of said fusion protein administered for a second period of time, and a third dose of between 0.1 mg/kg and 1.0 mg/kg of the ActRIIA fusion protein subsequently administered for a third period of time. In some embodiments, the first dose of ActRIIA fusion protein is administered to a patient in an amount from about 0.2 mg/kg to about 0.4 mg/kg. In some embodiments, the first dose of ActRIIA fusion protein is administered to the patient at a dose of 0.3 mg/kg. In some embodiments, the second dose of ActRIIA fusion protein is administered to the patient in an amount from about 0.5 mg/kg to about 0.8 mg/kg. In some embodiments, the second dose of ActRIIA fusion protein is administered to the patient at a dose of 0.7 mg/kg. In some embodiments, the third dose of ActRIIA fusion protein is administered to the patient in an amount from about 0.2 mg/kg to about 0.4 mg/kg. In some embodiments, the third dose of ActRIIA fusion protein is administered to the patient at a dose of 0.3 mg/kg.

In some embodiments, the dosing regimen comprises administering a first dose of an ActRIIA fusion protein to a patient in an amount of 0.3 mg/kg followed by administration of a second dose of an ActRIIA fusion protein to the patient in an amount of 0.7 mg/kg.

In some embodiments, the dosing regimen comprises administering a first dose of an ActRIIA fusion protein to a patient in an amount of 0.3 mg/kg, administering a second dose of an ActRIIA fusion protein to the patient in an amount of 0.7 mg/kg and administering all subsequent doses to the patient in an amount of 0.7 mg/kg.

In some embodiments, the dosing regimen comprises administering a first dose of an ActRIIA fusion protein to a patient in an amount of 0.3 mg/kg, administering a second dose of the ActRIIA fusion protein to the patient in an amount of 0.7 mg/kg, and administering a third dose of the ActRIIA fusion protein to the patient in an amount of 0.3 mg/kg.

In some embodiments, the second dose exceeds the first dose. In some embodiments, the first dose exceeds the second dose. In some embodiments, the third dose exceeds the second dose. In some embodiments, the second dose exceeds the third dose. In some embodiments, the first period of time is at least 3 weeks. In some embodiments, the second period of time is at least 3 weeks. In some embodiments, the third period of time is at least 3 weeks. In some embodiments, the second period of time is at least 21 weeks. In some embodiments, the second period of time is at least 45 weeks. In some embodiments, the second period of time exceeds the first period of time. In some embodiments, the third period of time exceeds the first period of time. In some embodiments, the third period of time exceeds the second period of time.

In some embodiments, the dosing regimen prevents, ameliorates, or decreases adverse effects of the ActRIIA fusion protein. In some embodiments, administration of an ActRIIA fusion protein in accordance with the dosage regimen as provided herein results in decreased adverse side effects.

In some embodiments, the methods of the invention comprise administering an ActRIIA fusion protein to a patient, wherein the dosing range of the ActRIIA fusion protein is 0.1 mg/kg (i.e. 0.1 mg of ActRIIA fusion protein per kg of a patient's body weight) to 2.0 mg/kg. In some embodiments, the ActRIIA fusion protein is administered at 0.1 mg/kg. In some embodiments, the ActRIIA fusion protein is administered at 0.2 mg/kg. In some embodiments, the ActRIIA fusion protein is administered at 0.3 mg/kg. In some embodiments, the ActRIIA fusion protein is administered at 0.4 mg/kg. In some embodiments, the ActRIIA fusion protein is administered at 0.5 mg/kg. In some embodiments, the ActRIIA fusion protein is administered at 0.6 mg/kg. In some embodiments, the ActRIIA fusion protein is administered at 0.7 mg/kg. In some embodiments, the ActRIIA fusion protein is administered at 0.8 mg/kg. In some embodiments, the ActRIIA fusion protein is administered at 0.9 mg/kg. In some embodiments, the ActRIIA fusion protein is administered at 1.0 mg/kg. In some embodiments, the ActRIIA fusion protein is administered at 1.1 mg/kg. In some embodiments, the ActRIIA fusion protein is administered at 1.2 mg/kg. In some embodiments, the ActRIIA fusion protein is administered at 1.3 mg/kg. In some embodiments, the ActRIIA fusion protein is administered at 1.4 mg/kg. In some embodiments, the ActRIIA fusion protein is administered at 1.5 mg/kg. In some embodiments, the ActRIIA fusion protein is administered at 1.6 mg/kg. In some embodiments, the ActRIIA fusion protein is administered at 1.7 mg/kg. In some embodiments, the ActRIIA fusion protein is administered at 1.8 mg/kg. In some embodiments, the ActRIIA fusion protein is administered at 1.9 mg/kg. In some embodiments, the ActRIIA fusion protein is administered at 2.0 mg/kg.

In certain embodiments, the methods of the invention comprise administering an ActRIIA fusion protein to a patient once a day. In certain embodiments, the ActRIIA fusion protein is administered twice a day. In certain embodiments, the ActRIIA fusion protein is administered once a week. In certain embodiments, the ActRIIA fusion protein is administered twice a week. In certain embodiments, the ActRIIA fusion protein is administered three times a week. In certain embodiments, the ActRIIA fusion protein is administered once every two weeks. In certain embodiments, the ActRIIA fusion protein is administered once every three weeks. In certain embodiments, the ActRIIA fusion protein is administered once every four weeks. In certain embodiments, the ActRIIA fusion protein is administered once per month.

### Compositions and Kits

The disclosure provides a kit comprising a lyophilized fusion protein and an injection device and instructions for carrying out a method of treating a patient with PAH, as described herein. In certain embodiments, the lyophilized fusion protein comprises an ActRIIA fusion protein (e.g., an ActRIIA fusion protein that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 4 or SEQ ID NO: 5), or fragments, functional variants, or modified forms thereof.

In some embodiments, the lyophilized fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 1. In certain embodiments, the fusion protein comprises a polypeptide that consists of the amino acid sequence of SEQ ID NO: 1.

In certain embodiments of the foregoing, the lyophilized fusion protein of SEQ ID NO: 1 comprises a fusion protein further comprising an Fc domain of an immunoglobulin. In certain such embodiments, the Fc domain of the immunoglobulin is an Fc domain of an IgG1 immunoglobulin. In certain embodiments, the Fc domain comprises the amino acid sequence of SEQ ID NO: 3. In certain embodiments, the Fc domain is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO: 3.

In other embodiments, the fusion protein further comprises a linker domain positioned between the polypeptide domain and the Fc domain of the immunoglobulin. In certain embodiments, the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21). In certain embodiments, the linker domain comprises or consists of TGGG (SEQ ID NO: 20).

In certain embodiments, the lyophilized fusion protein is part of a homodimer protein complex.

In certain embodiments, the fusion protein is glycosylated.

In some embodiments, a composition comprising an ActRIIA fusion protein is provided as a liquid formulation or prepared by reconstituting a lyophilized powder with sterile water for injection prior to use.

The disclosure also provides a kit comprising a sterile powder comprising a lyophilized fusion protein as disclosed herein, an injection device, and instructions for carrying out a method of the disclosure. In some embodiments of the kits disclosed herein, the sterile powder comprising a lyophilized fusion protein is pre-filled in one or more containers, such as one or more vials.

In certain embodiments, the pH range of the sterile powder comprising a lyophilized fusion protein, once reconstituted, is from 7 to 8. In some embodiments, the sterile powder comprising a lyophilized fusion protein further comprises a buffering agent. In some embodiments, once the sterile powder is reconstituted, the buffering agent is present in an amount of at least 10 mM. In some embodiments, the buffering agent is present in an amount in the range of between about 10 mM to about 200 mM. In some embodiments, the buffering agent comprises citric acid monohydrate and/or trisodium citrate dehydrate.

In some embodiments, the sterile powder comprising a lyophilized fusion protein further comprises a surfactant. In some embodiments, the surfactant comprises a polysorbate. In some embodiments, the surfactant comprises polysorbate 80.

In some embodiments, the sterile powder comprising a lyophilized fusion protein further comprises a lyoprotectant. In some embodiments, the lyoprotectant comprises a sugar, such as disaccharides (e.g, sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the sterile powder comprises the lyoprotectant and lyophilized fusion protein in a weight ratio of at least 1:1 lyophilized fusion protein to lyoprotectant. In some embodiments, the sterile powder comprises the lyoprotectant and lyophilized fusion protein in a weight ratio of from 1:1 to 1:10 lyophilized fusion protein to lyoprotectant. In some embodiments, the sterile powder comprises the lyoprotectant and lyophilized fusion protein in a weight ratio of 1:1, 1:2, 1:3, 1:4, 1:5, 1:6, 1:7, 1:8, 1:9, or 1:10 lyophilized fusion protein to lyoprotectant. In some embodiments, the sterile powder comprises the lyoprotectant and lyophilized fusion protein in a weight ratio of 1:6 lyophilized fusion protein to lyoprotectant. In certain embodiments of the foregoing, the sterile powder comprises lyoprotectant in an amount sufficient to stabilize the lyophilized fusion protein.

In certain embodiments of the kits disclosed herein, the injection device comprises a syringe. In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises aqueous solutions such as water, physiologically buffered saline, or other solvents or vehicles such as glycols, glycerol, oils or injectable organic esters. In some embodiments, the pharmaceutically acceptable excipient comprises a pharmaceutically acceptable excipient selected from calcium phosphates, calcium carbonates, calcium sulfates, halites, metallic oxides, sugars, sugar alcohols, starch, glycols, povidones, mineral hydrocarbons, acrylic polymers, fatty alcohols, mineral stearates, glycerin, and/or lipids. In certain embodiments, the reconstitution solution comprises pharmaceutically acceptable sterile isotonic aqueous or nonaqueous solutions, dispersions, suspensions or emulsions. In certain such embodiments, the reconstitution solution comprises antioxidants, buffers, bacteriostats, and/or solutes which render the formulation isotonic with the blood of the intended recipient. In other embodiments, the reconstitution solution comprises suspending or thickening agents.

In certain embodiments of the kits disclosed herein, the kit further comprises a vial adapter. In some embodiments, the vial pre-filled with sterile powder comprising a lyophilized fusion protein attaches to one end of the vial adapter. In some embodiments, the syringe pre-filled with a reconstitution solution as disclosed herein attaches to an end of the vial adapter. In some embodiments, the syringe pre-filled with a reconstitution solution as disclosed herein and the vial pre-filled with sterile powder comprising a lyophilized fusion protein are attached to opposite ends of the vial adapter. In some embodiments, the reconstitution solution is transferred from the pre-filled syringe to the vial. In some embodiments, transfer of the reconstitution solution to the vial pre-filled with sterile powder comprising a lyophilized fusion protein reconstitutes the lyophilized fusion protein into a sterile injectable solution. In some embodiments, the lyophilized fusion protein is reconstituted into a sterile injectable solution. In some embodiments, the lyophilized fusion protein is reconstituted into a sterile injectable solution prior to use.

The disclosure also provides a kit used for reconstituting a lyophilized fusion protein into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose. In some embodiments, the weight based dose is 0.3 mg/kg. In some embodiments, the weight based dose is 0.7 mg/kg.

In some embodiments of the kits disclosed herein, the kit further comprises one or more vials or cartridges containing the lyophilized fusion protein. In some embodiments, the kit comprises at least two vials or cartridges containing the lyophilized fusion protein. In some embodiments, the kit comprises at least three vials or cartridges containing the lyophilized fusion protein. In some embodiments, the two vials can contain the same or different amounts of the lyophilized fusion protein. In some embodiments, the vials or cartridges comprise a vial or cartridge containing between 25 mg to 60 mg of lyophilized fusion protein. In some embodiments, at least one of the vials or cartridges comprise a vial or cartridge containing 60 mg of lyophilized fusion protein. In some embodiments, at least one of the vials or cartridges comprise a vial or cartridge containing 45 mg of lyophilized fusion protein. In some embodiments, at least one of the vials or cartridges comprise a vial or cartridge containing 30 mg of lyophilized fusion protein. In some embodiments, at least one of the vials or cartridges comprise a vial or cartridge containing 25 mg of lyophilized fusion protein. In some embodiments, a first vial or cartridge contains 45 mg of lyophilized fusion protein and a second vial or cartridge contains 60 mg of lyophilized fusion protein. In some embodiments, a first vial or cartridge contains 30 mg of lyophilized fusion protein and a second vial or cartridge contains 60 mg of lyophilized fusion protein. In some embodiments, a first vial or cartridge contains 30 mg of lyophilized fusion protein, a second vial or cartridge contains 45 mg of lyophilized fusion protein, and a third vial or cartridge contains 60 mg of lyophilized fusion protein. In some embodiments, a first vial or cartridge contains 25 mg of lyophilized fusion protein, a second vial or cartridge contains 45 mg of lyophilized fusion protein, and a third vial or cartridge contains 60 mg of lyophilized fusion protein. In some embodiments, the one or more vials or cartridges are refrigerated at 2-8° C.

In certain embodiments, the kits described herein comprise a liquid formulation of 10-100 mg/mL of an ActRIIA fusion protein, a buffer, a surfactant, a stabilizing agent, and optionally one or more antioxidants, wherein the buffer is not histidine, and instructions for carrying out a method of the invention.

In certain embodiments, the kits described herein comprise a liquid formulation of 10-100 mg/mL of an ActRIIA fusion protein of SEQ ID NO: 4 and/or SEQ ID NO:5, a buffer, a surfactant, a stabilizing agent, and optionally one or more antioxidants, wherein the buffer is not histidine, and instructions for carrying out a method of the invention.

In certain embodiments, the kits described herein comprise a liquid formulation of 10-100 mg/mL of an ActRIIA fusion protein of SEQ ID NO: 4, a buffer, a surfactant, a stabilizing agent, and optionally one or more antioxidants, wherein the buffer is not histidine, and instructions for carrying out a method of the invention.

In certain embodiments, the kits described herein comprise a liquid formulation of 10-100 mg/mL of an ActRIIA fusion protein of SEQ ID NO: 5, a buffer, a surfactant, a stabilizing agent, and optionally one or more antioxidants, wherein the buffer is not histidine, and instructions for carrying out a method of the invention.

In certain embodiments, the kits described herein comprise a liquid formulation of an ActRIIA fusion protein in an auto-injector and instructions for carrying out a method of the invention.

### EXAMPLES

The following examples are meant to be illustrative and should not be construed as further limiting. The contents of the figures and all references, patents, and published patent applications cited throughout this application are expressly incorporated herein by reference.

### Example 1

Effects of Sotatercept, an Activin Signalling Inhibitor, on Lung Diffusion Capacity for Carbon Monoxide (DLCO) and Blood Gases in Patients with Pulmonary Arterial Hypertension (PAH)

DLCO and arterialized capillary blood gases (arterial oxygen tension [PaO₂], arterial carbon dioxide tension [PaCO₂], and arterial oxygen saturation [SaO₂]) were assessed in patients with PAH who were enrolled into two randomized, double-blind, placebo-controlled trials with sotatercept (PULSAR and STELLAR). Measurements were obtained during the screening period and after 24 weeks into the study when participants and members of the study team were still blinded to the assigned treatment.

All patients enrolled into PULSAR and STELLAR were included in the present analysis. Both studies enrolled pre-treated patients with PAH with a pulmonary vascular resistance (PVR)≥5 Wood units (WU) at screening in whom relevant cardiopulmonary co-morbidities had been excluded. In PULSAR, participants were randomized 1:1:1 to receive placebo or sotatercept at doses of 0.3 mg/kg or 0.7 mg/kg every 3 weeks. In STELLAR, participants were randomized 1:1 to receive placebo or sotatercept at a target dose of 0.7 mg/kg every 3 weeks. Protocol assessments included right heart catheterization, 6-minute walk testing, and measurements of NT-proBNP during the 4-week screening periods and after 24 weeks.

After completion of the 24-week study period, patients who participated in PULSAR were eligible to roll over into an open-label extension study, in which they received sotatercept at doses of 0.3 mg/kg or 0.7 mg/kg every 3 weeks. In STELLAR, patients who had a clinical worsening event after week 24 were allowed to switch into the open-label SOTERIA study (clinicaltrials.gov identifier NCT04796337), where they received sotatercept at a target dose of 0.7 mg/kg. All other patients rolled over into the SOTERIA study upon STELLAR completion in October/November 2022.

Pulmonary function testing with DLCO measurements and blood gas analyses were obtained during the screening period of the studies, i.e., within 4 weeks of randomization, and at week 24.

The DLCO was measured with the single breath technique using a commercially available device (Ganshorn, Niederlauer, Germany). The DLCO values reported in this study have been corrected for the actual haemoglobin (Hb) value using the following equation:

DLCO_{[corrected for Hb]}=DLCO_{[measured]}·(1.7 Hb/(10.22+Hb)).

Blood gas analyses from arterialized capillary blood (taken from hyperaemized earlobes) were obtained while the patients were resting in a sitting position and breathing ambient air for at least 10 min. The samples were analyzed without delay using a commercially available device (Radiometer, Copenhagen, Denmark).

Variables of interest for the present analyses were DLCO (expressed as percentage of the predicted value), arterial oxygen tension (PaO₂), arterial carbon dioxide tension (PaCO₂), and arterial oxygen saturation (SaO₂). In addition, changes from baseline at week 24 in haemoglobin values, 6-minute walking distance (6MWD), WHO functional class (FC), NT-proBNP, and haemodynamics were captured.

As secondary analysis, the changes in DLCO and blood gases in patients who had originally been assigned to the placebo arms 12-24 weeks after transition to the PULSAR open-label extension study or open-label SOTERIA study were assessed, respectively.

### Statistical Analysis

Categorical data are presented as N (%) and continuous data as mean±standard deviation (SD). Group comparisons at baseline were made with Fisher's exact test or Pearson's Chi-squared test for categorical data and with Wilcoxon's rank sum test for continuous variables. Changes from baseline at week 24 in the respective treatment groups are also presented as mean and 95% confidence interval (CI). Between-group differences of changes from baseline at week 24 are shown as mean and 95% CI. Values obtained at baseline and week 24 in the respective treatment arms were compared with the Wilcoxon signed-rank test. Between-group comparisons were conducted using a Wilcoxon rank sum test. P-values ≤0.05 were considered statistically significant.

### Results

A total of 35 patients were enrolled into the sotatercept studies, of whom 20 were assigned to the placebo group and 15 to the sotatercept group. The mean difference in the change from baseline at week 24 in DLCO (expressed as percentage of the predicted value) between the placebo group and the sotatercept group was 8% (95% confidence interval [CI], 4 to 11; p<0.001), favoring sotatercept. The mean difference between the two groups in the change from baseline at week 24 in PaO₂ was 5 mmHg (95% CI, 1 to 10; p=0.026), also favoring sotatercept. No significant differences between the two groups were seen in changes from baseline at week 24 in PaCO₂ and SaO₂.

Between Feb. 5, 2019 and Jun. 4, 2019, 7 patients with PAH were enrolled into the PULSAR study. Between Jul. 16, 2021 and Feb. 10, 2022, 28 patients were enrolled into the STELLAR study. Of these 35 patients, 20 were assigned to the respective placebo group and 15 to the respective sotatercept group. All patients were retained in the studies throughout the 24-weeks study periods, except one. This patient discontinued from STELLAR because she became pregnant but was kept in the current analysis as she had been randomized to receive placebo and did not undergo changes in her PAH medication during the observation period (the pregnancy was terminated). This patient was excluded from the secondary analysis of changed in DLCO and blood gases after switching from placebo to active treatment.

Patient demographics and characteristics at baseline were comparable across both groups showing a relatively young patient population consisting mostly of patients with idiopathic and heritable PAH who were treated with double or triple combinations of currently available PAH medications (Table 1).

TABLE 1
Patient characteristics and treatments for pulmonary
arterial hypertension at enrollment

| | All | | Placebo | | Sotatercept | | |
|---|---|---|---|---|---|---|---|
| | N = 35 | | N = 20 | | N = 15 | | p |
| Age (years) | 44.7 ± 13.6 | | 44.1 ± 11.9 | | 45.4 ± 15.9 | | 0.881 |
| Female sex, N (%) | 28 | (80%) | 16 | (80%) | 12 | (80%) | 1.000* |
| BMI (kg/m²) | 26.4 ± 6.5 | | 26.2 ± 5.7 | | 26.7 ± 7.6 | | 0.726 |
| PAH subtype | | | | | | | |
| Idiopathic, N (%) | 23 | (66%) | 13 | (65%) | 10 | (67%) | 0.673* |
| Heritable, N (%) | 8 | (23%) | 5 | (25%) | 3 | (20%) | |
| Drug-related, N (%) | 1 | (3%) | 0 | (0%) | 1 | (7%) | |
| CHD, N (%) | 3 | (9%) | 2 | (10%) | 1 | (7%) | |
| WHO FC | | | | | | | |
| I, N (%) | 0 | (0%) | 0 | (0%) | 0 | (0%) | 0.589* |
| II, N (%) | 10 | (29%) | 5 | (25%) | 5 | (33%) | |
| III, N (%) | 25 | (71%) | 15 | (75%) | 10 | (67%) | |
| IV, N (%) | 0 | (0%) | 0 | (0%) | 0 | (0%) | |
| 6MWD, m | 423 ± 64 | | 432 ± 53 | | 411 ± 76 | | 0.433 |
| NT-proBNP (ng/L) | 1584 ± 1916 | | 1622 ± 1780 | | 1533 ± 2145 | | 0.764 |
| TLC, % pred | 93 ± 13 | | 92 ± 13 | | 95 ± 12 | | 0.648 |
| FVC, % pred | 81 ± 15 | | 80 ± 12 | | 84 ± 18 | | 0.473 |
| FEV_{1/}FVC, % | 76 ± 8 | | 74 ± 9 | | 77 ± 7 | | 0.405 |
| DLCO, % predicted | 62 ± 14 | | 62 ± 12 | | 62 ± 16 | | 0.881 |
| PaO₂, mmHg | 67 ± 10 | | 68 ± 11 | | 66 ± 8 | | 0.973 |
| PaCO₂, mmHg | 31 ± 4 | | 31 ± 3 | | 32 ± 4 | | 0.960 |
| SaO₂, % | 94 ± 2 | | 94 ± 2 | | 94 ± 2 | | 0.880 |
| RAP, mmHg | 9 ± 3 | | 10 ± 4 | | 9 ± 3 | | 0.502 |
| mPAP, mmHg | 60 ± 12 | | 59 ± 10 | | 62 ± 15 | | 0.504 |
| PAWP, mmHg | 9 ± 3 | | 9 ± 3 | | 9 ± 3 | | 0.841 |
| Cardiac index, | 2.5 ± 0.5 | | 2.5 ± 0.4 | | 2.5 ± 0.6 | | 0.725 |
| L/min/m² | | | | | | | |
| PVR, WU | 12.0 ± 5.6 | | 11.6 ± 4.8 | | 12.6 ± 6.8 | | 0.739 |
| SvO₂, % | 65 ± 7 | | 65 ± 7 | | 66 ± 7 | | 0.790 |
| Dual oral | 9 | (26%) | 4 | (20%) | 5 | (33%) | 0.451# |
| combination | | | | | | | |
| therapy, N (%)** | | | | | | | |
| Triple therapy, | 26 | (74%) | 16 | (80%) | 10 | (66%) | 0.451# |
| N (%)*** | | | | | | | |
| Triple therapy | 20 | (57%) | 13 | (65%) | 7 | (47%) | 0.321# |
| including SC or IV | | | | | | | |
| treprostinil, N (%) | | | | | | | |

*Chi²;
# Fisher's Exact
**Dual oral combination therapy consists of an endothelin receptor antagonist and a phosphodiesterase-5 inhibitor or a soluble guanylate cyclase stimulator
***Triple therapy consists of an endothelin receptor antagonist, a phosphodiesterase-5 inhibitor or a soluble guanylate cyclase stimulator, and a prostacyclin analogue or a prostacyclin receptor agonist

At week 24, there were improvements in cardiopulmonary haemodynamics, including a placebo-adjusted decline of 19 mmHg in mean pulmonary artery pressure (mPAP), a placebo-adjusted decline of 5.9 WU in PVR, and a placebo-adjusted increase in cardiac index of 0.3 L/min/m2. In the sotatercept group, haemoglobin increased by 1.3 d/dl, and the mixed venous oxygen saturation (SvO₂) increased by 4.7%. Details are shown in Table 2. Table 2: Changes in haemoglobin, 6-minute walk distance, NT-proBNP, and haemodynamics 24 weeks after treatment initiation

| | Placebo | Sotatercept |
|---|---|---|
| | N = 20 | N = 15 |
| Haemoglobin (g/dl) Mean | −0.0 (95% | 1.3 (95% CI |
| (95% CI) change from baseline | CI −0.6 | 0.7 to 1.9) |
| | to 0.5 | |

| | |
|---|---|
| Between group comparison, | 1.3 (95% CI 0.5 to 2.2); |
| mean (95% CI) | p = 0.002 |

| | | |
|---|---|---|
| 6 min walk distance (m) Mean | −44 (95% | 72 (95% CI |
| (95% CI) change from baseline | CI −74 | 37 to 109) |
| | to −13) | |

| | |
|---|---|
| Between group comparison, | 117 (95% CI 69 to |
| mean (95% CI) | 164); p < 0.001 |

| | | |
|---|---|---|
| NT-proBNP (ng/L) Mean (95% | 671 (95% | −1209 (95% |
| CI) change from baseline | CI −163 | CI −2172 |
| | to 1505) | to −246) |

| | |
|---|---|
| Between group comparison, | −1881 (95% CI −3155 |
| mean (95% CI) | to −607); p = 0.005 |

| | | |
|---|---|---|
| Right atrial pressure (mmHg) | 0.4 (95% | −3.6 (95% |
| Mean (95% CI) change from | CI −1.0 | CI −5.2 |
| baseline | to 1.7) | to −2.0) |

| | |
|---|---|
| Between group comparison, | −4 (95% CI −6.1 |
| mean (95% CI) | to −1.8); p < 0.001 |

| | | |
|---|---|---|
| Mean pulmonary arterial | 1 (95% | −19 (95% |
| pressure (mmHg) Mean (95% | CI −4 | CI −24 |
| CI) change from baseline | to 5) | to −13) |

| | |
|---|---|
| Between group comparison, | −19 (95% CI −25 |
| mean (95% CI) | to −13); p < 0.001 |

| | | |
|---|---|---|
| Pulmonary capillary wedge | −0.2 (95% | −1.8 (95% |
| pressure (mmHg) Mean (95% | CI −1.7 | CI −3.6 |
| CI) change from baseline | to 1.4) | to −0.0) |

| | |
|---|---|
| Between group comparison, | −1.7 (95% CI −4.0 |
| mean (95% CI) | to 0.7), p = 0.161 |

| | | |
|---|---|---|
| Cardiac index (L/min) Mean | −0.2 (95% | 0.1 (95% |
| (95% CI) change from baseline | CI −0.4 | CI −0.1 |
| | to −0.0) | to 0.4) |

| | |
|---|---|
| Between group comparison, | 0.3 (95% CI 0.03 to |
| mean (95% CI) | 0.6), p = 0.070 |

| | | |
|---|---|---|
| Pulmonary vascular resistance | 0.9 (95% | −4.8 (95% |
| (Wood units) Mean (95% CI) | CI −0.8 | CI −7.0 |
| change from baseline | to 3.1) | to −2.6) |

| | |
|---|---|
| Between group comparison, | −5.9 (95% CI −8.9 |
| mean (95% CI) | to −3.0), p < 0.001 |

| | | |
|---|---|---|
| Mixed venous oxygen | −1.5 (95% | 4.7 (95% |
| saturation (%) Mean (95% CI) | CI −3.8 | CI 2.0 |
| change from baseline | to 0.8) | to 7.4) |

| | |
|---|---|
| Between group comparison, | 6.2 (95% CI 2.5 to 9.7), |
| mean (95% CI) | p = 0.001 |

Abbreviations: NT-proBNP, N-terminal fragment of pro-brain natriuretic peptide; CI, confidence interval.

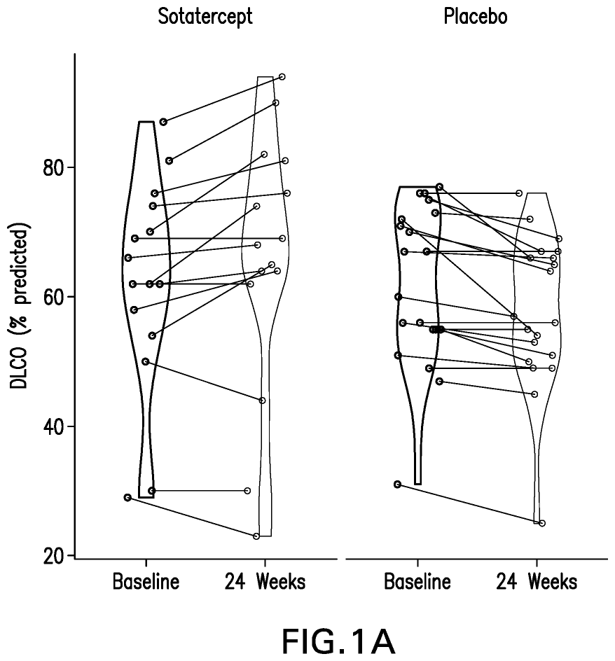

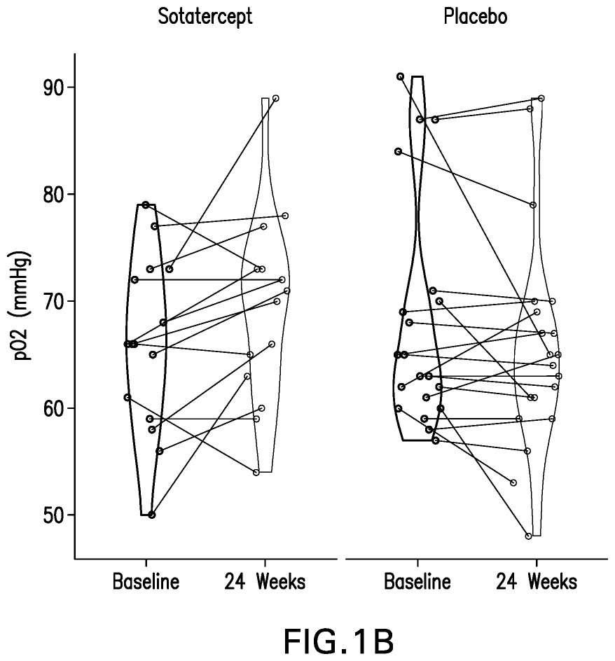

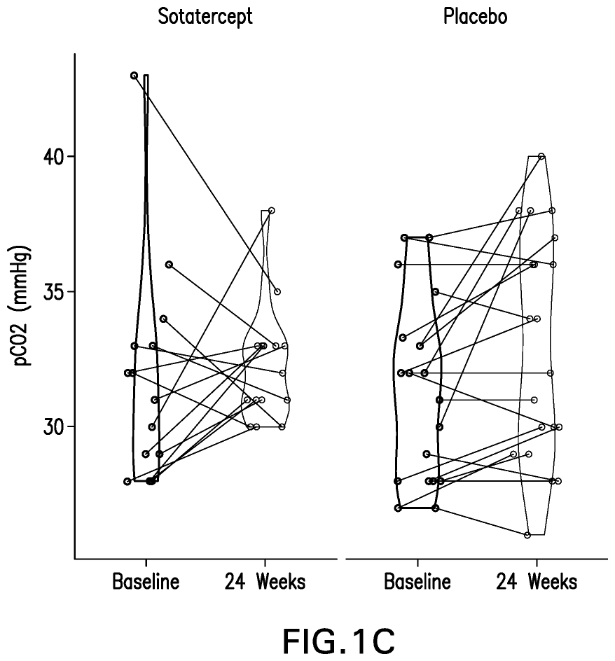

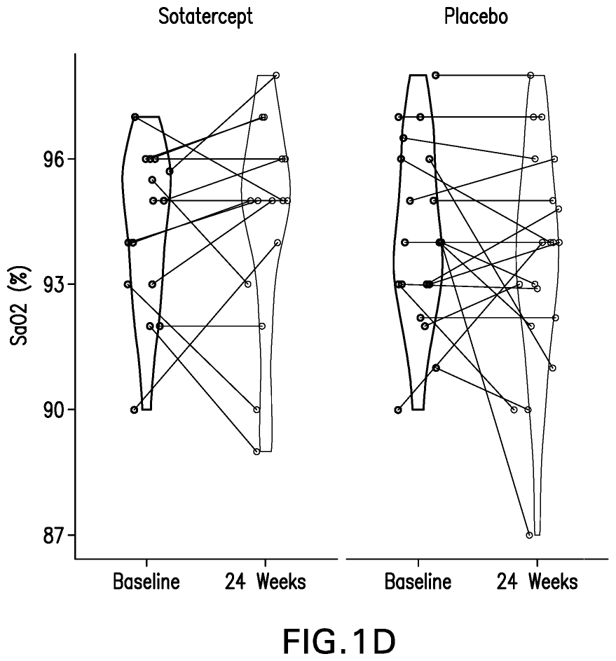

The changes from baseline at week 24 in DLCO and blood gases in the two patient cohorts are shown in Table 3 and FIG. 1.

TABLE 3
Changes in DLCO and blood gases 24
weeks after treatment initiation

| | Placebo | Sotatercept |
|---|---|---|
| | N = 20 | N = 15 |
| DLCO (% of the predicted | −4 (95% | 4 (95% |
| value) Mean (95% CI) | CI, −6 | CI, 1 to 6) |
| change from baseline | to −2) | |

| | |
|---|---|
| Between group comparison, | 8 (95% CI, 4 to 11); |
| mean (95% CI) | p < 0.001 |

| | | |
|---|---|---|
| PaO₂ (mmHg) Mean (95% | −2 (95% | 3 (95% |
| CI) change from baseline | CI, −5 to 1) | CI, 0 to 7) |

| | |
|---|---|
| Between group comparison, | 5 (95% CI 1 to 10); |
| mean (95% CI) | p = 0.026 |

| | | |
|---|---|---|
| PaCO₂ (mmHg) Mean (95% | 2 (95% | 1 (95% |
| CI) change from baseline | CI, 0 to 3) | CI, −1 to 3) |

| | |
|---|---|
| Between group comparison, | −1 (95% CI −3 to 2); |
| mean (95% CI) | p = 0.531 |

| | | |
|---|---|---|
| SaO₂ (mmHg) Mean (95% | 0 (95% | 0 (95% |
| CI) change from baseline | CI, −2 to 1) | CI, −1 to 2) |

| | |
|---|---|
| Between group comparison, | 1 (95% CI −1 to 2); |
| mean (95% CI) | p = 0.583 |

The DLCO of the placebo group decreased by 4% (95% CI, −6 to −2) while the DLCO of the sotatercept group increased by 4% (95% CI, 1 to 6). The mean difference in the DLCO change between the two groups was 8% (95% CI, 4 to 11; p<0.001). PaO₂ improved as well with sotatercept compared to placebo, while there were no significant changes in PaCO₂ and SaO₂.

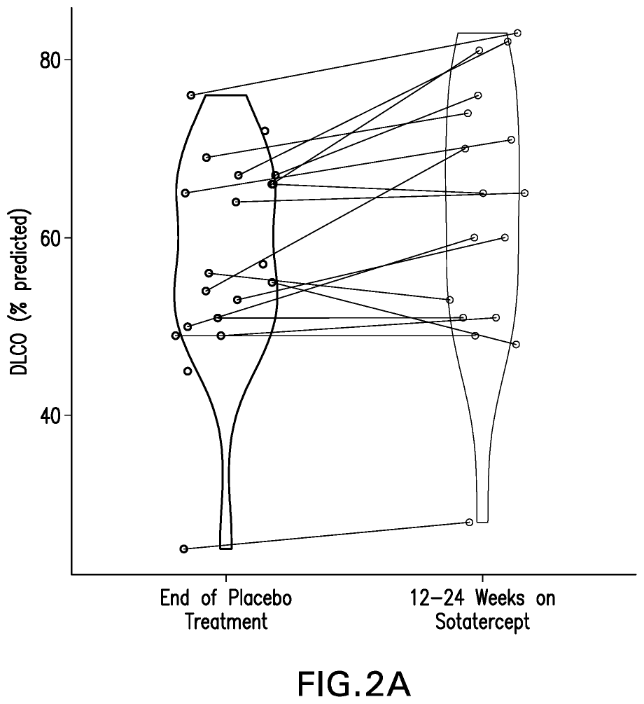

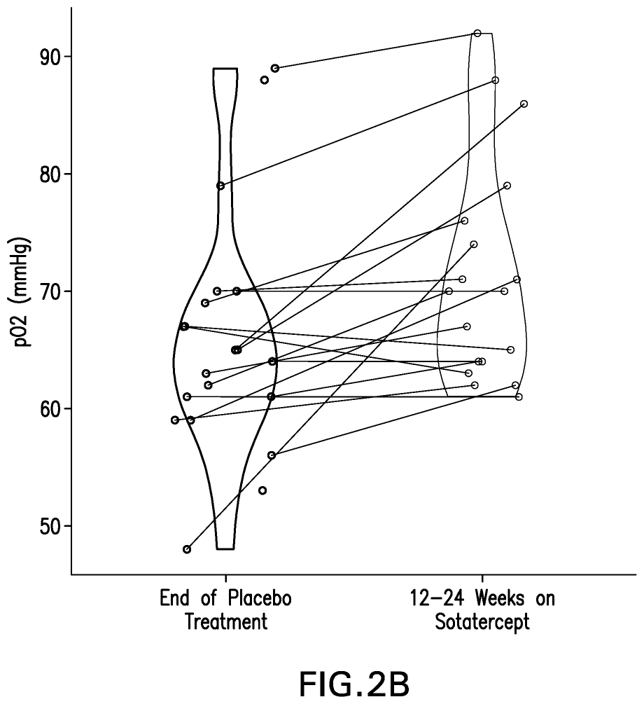

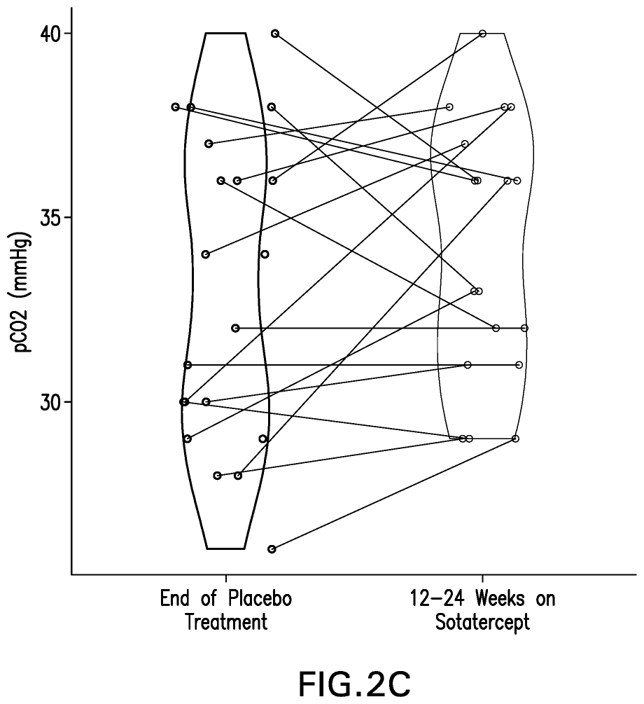

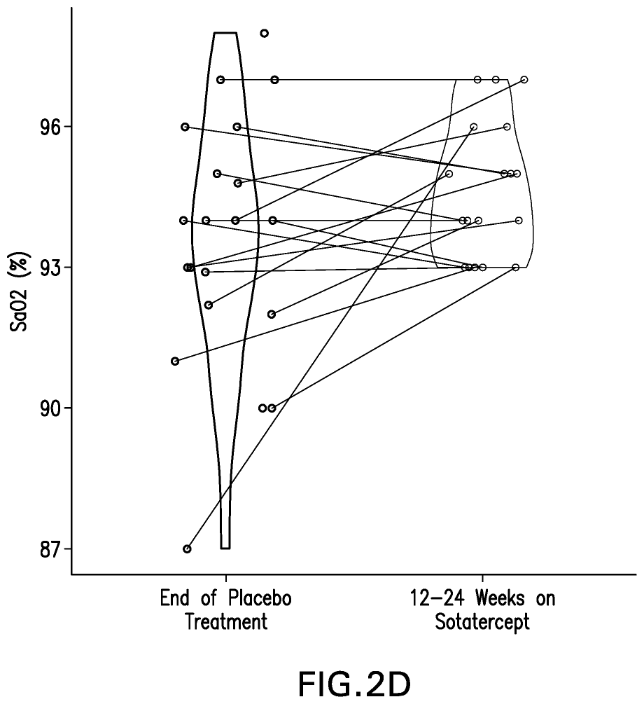

The secondary analysis of patients who were originally assigned to the placebo arm of their respective studies showed improvements in DLCO and PaO₂ within 12-24 weeks after initiation of sotatercept therapy. The improvements were comparable to those observed in patients originally assigned to active therapy: The DLCO increased by 3% (95% CI, −1 to 7) and the PaO₂ increased by 7 mmHg (95% CI, 3 to 12). PaCO₂ and SaO₂ changed by 1 mmHg (95% CI, −1 to 3) and 2% (95% CI, 0 to 3), respectively, as shown in FIG. 2. FIG. 2 shows DLCO, PaO₂, PaCO₂ and SaO₂ in patients who were initially assigned to the placebo arms of the respective studies obtained at the end of placebo treatment and at 12-24 weeks on sotatercept.

Data suggest that sotatercept may improve lung diffusion capacity and gas exchange in patients with PAH. The observation that sotatercept resulted in an improved PaO₂ is of clinical interest as well. The patients participating in this analysis had mildly reduced PaO₂ at baseline, which is a characteristic finding in patients with PAH. Hence, the oxygen binding curve of these patients lay in the upper flat region, so that the improved PaO₂ did not translate into an improved SaO₂. Nevertheless, the improved PaO₂ might become clinically relevant in patients who present with more profound hypoxemia.

The mechanisms resulting in an increased PaO₂ are probably multifactorial. Improved diffusion capacity could be a contributing factor. At the same time, SvO₂ improved as well, presumably resulting from both the mild but statistically significant improvement in cardiac output and from the increase in haemoglobin, the latter being a well-known effect of sotatercept, which was originally developed to increase haemoglobin levels in patients with oncologic conditions. The increase in haemoglobin together with the slight increase in cardiac output and the unchanged or mildly improved SaO₂ results in an increased oxygen delivery, which could explain why the placebo adjusted SvO₂ improved by 6%, which was larger than what would have been expected from the increase in cardiac output. In the STELLAR trial, there was a placebo-corrected improvement in SvO₂ by 4% while the cardiac output did not change, a finding that would also be explainable by improved oxygen delivery owing to an increase in haemoglobin.

It was observed that PaCO₂ did not change with sotatercept treatment. A low PaCO₂ at rest and during exercise is a well-described phenomenon in patients with PAH and has been linked to adverse outcomes. The mean baseline PaCO₂ of 31 mmHg in the present series was in line with previous publications.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

| SEQ ID NO: | Sequence |
|---|---|
| 1 | ILGRSETQECLFFNANWEKDRTNQTGVEPCYGDKDK |
| | RRHCFATWKNISGSIEIVKQGCWLDDINCYDRTDCVE |
| | KKDSPEVYFCCCEGNMCNEKFSYFPEMEVTQPTSNP |
| | VTPKPP |
| 2 | TGGG |
| 3 | THTCPPCPAPELLGGPSVFLFPPKPKDTLMISRTPEVT |
| | CVVVDVSHEDPEVKFNWYVDGVEVHNAKTKPREEQ |
| | YNSTYRVVSVLTVLHQDWLNGKEYKCKVSNKALPV |
| | PIEKTISKAKGQPREPQVYTLPPSREEMTKNQVSLTCL |
| | VKGFYPSDIAVEWESNGQPENNYKTTPPVLDSDGSFF |
| | LYSKLTVDKSRWQQGNVFSCSVMHEALHNHYTQKS |
| | LSLSPGK |
| 4 | ILGRSETQECLFFNANWEKDRTNQTGVEPCYGDKDK |
| | RRHCFATWKNISGSIEIVKQGCWLDDINCYDRTDCVE |
| | KKDSPEVYFCCCEGNMCNEKFSYFPEMEVTQPTSNP |
| | VTPKPPTGGGTHTCPPCPAPELLGGPSVFLFPPKPKDT |
| | LMISRTPEVTCVVVDVSHEDPEVKFNWYVDGVEVH |
| | NAKTKPREEQYNSTYRVVSVLTVLHQDWLNGKEYK |
| | CKVSNKALPVPIEKTISKAKGQPREPQVYTLPPSREE |
| | MTKNQVSLTCLVKGFYPSDIAVEWESNGQPENNYKT |
| | TPPVLDSDGSFFLYSKLTVDKSRWQQGNVFSCSVMH |
| | EALHNHYTQKSLSLSPGK |
| 5 | ILGRSETQECLFFNANWEKDRTNQTGVEPCYGDKDK |
| | RRHCFATWKNISGSIEIVKQGCWLDDINCYDRTDCVE |
| | KKDSPEVYFCCCEGNMCNEKFSYFPEMEVTQPTSNP |
| | VTPKPPTGGGTHTCPPCPAPELLGGPSVFLFPPKPKDT |
| | LMISRTPEVTCVVVDVSHEDPEVKFNWYVDGVEVH |
| | NAKTKPREEQYNSTYRVVSVLTVLHQDWLNGKEYK |
| | CKVSNKALPVPIEKTISKAKGQPREPQVYTLPPSREE |
| | MTKNQVSLTCLVKGFYPSDIAVEWESNGQPENNYKT |
| | TPPVLDSDGSFFLYSKLTVDKSRWQQGNVFSCSVMH |
| | EALHNHYTQKSLSLSPG |
| 6 | TGGGG (SEQ ID NO: 6) |
| 7 | SGGGG |
| 8 | GGGGS |
| 9 | GGG |
| 10 | GGGG |
| 11 | SGGG |
| 12 | MGAAAKLAFAVFLISCSSGAILGRSETQECLFFNANW |
| | EKD |
| | RTNQTGVEPCYGDKDKRRHCFATWKNISGSIEIVKQ |
| | GCWLDDINCYDRTDCVEKKDSPEVYFCCCEGNMCN |
| | EKFSYFPEMEVTQPTSNPVTPKPPYYNILLYSLVPLML |
| | IAGIVICAFWV |
| | YRHHKMAYPPVLVPTQDPGPPPPSPLLGLKPLQLLEV |
| | KARGRFGCVWKAQLLNEYVAVKIFPIQDKQSWQNE |
| | YEVYSLPGMKHENILQFIGAEKRGTSVDVDLWLITAF |
| | HEKGSLSDFLKANVVSWNELCHIAETMARGLAYLHE |
| | DIPGLKDGHKPAISHRDIKSKNVLLKNNLTACIADFG |
| | LALKFEAGKSAGDTHGQVGTRRYMAPEVLEGAINFQ |
| | RDAFLRIDMYAMGLVLWELASRCTAADGPVDEYML |
| | PFEEEIGQHPSLEDMQEVVVHKKKRPVLRDYWQKH |
| | AGMAMLCETIEECWDHDAEARLSAGCVGERIT |
| | QMQRLTNIITTEDIVTVVTMVTNVDFPPKESSL |
| 13 | ILGRSETQECLFFNANWEKDRTNQTGVEPCYGDKDK |
| | RRHCFATWKNISGSIEIVKQGCWLDDINCYDRTDCVE |
| | KKDSPEVYFCCCEGNMCNEKFSYFPEM |
| 14 | atgggagctg ctgcaaagtt ggcgtttgcc gtctttctta tctcctgttc |
| | ttcaggtgct atacttggta gatcagaaac tcaggagtgt cttttcttta |
| | atgctaattg ggaaaaagac agaaccaatc aaactggtgt tgaaccgtgt |
| | tatggtgaca aagataaacg gcggcattgt tttgctacct ggaagaatat |
| | ttctggttcc attgaaatag tgaaacaagg ttgttggctg gatgatatca |
| | actgctatga caggactgat tgtgtagaaa aaaaagacag ccctgaagta |
| | tatttttgtt gctgtgaggg caatatgtgt aatgaaaagt tttcttattt |
| | tccggagatg gaagtcacac agcccacttc aaatccagtt acacctaagc |
| | caccctatta caacatcctg ctctattcct tggtgccact tatgttaatt |
| | gcggggattg tcatttgtgc attttgggtg tacaggcatc acaagatggc |
| | ctaccctcct gtacttgttc caactcaaga cccaggacca cccccacctt |
| | ctccattact aggtttgaaa ccactgcagt tattagaagt gaaagcaagg |
| | ggaagatttg gttgtgtctg gaaagcccag ttgcttaacg aatatgtggc |
| | tgtcaaaata tttccaatac aggacaaaca gtcatggcaa aatgaatacg |
| | aagtctacag tttgcctgga atgaagcatg agaacatatt acagttcatt |
| | ggtgcagaaa aacgaggcac cagtgttgat gtggatcttt ggctgatcac |
| | agcatttcat gaaaagggtt cactatcaga ctttcttaag gctaatgtgg |
| | tctcttggaa tgaactgtgt catattgcag aaaccatggc tagaggattg |
| | gcatatttac atgaggatat acctggccta aaagatggcc acaaacctgc |
| | catatctcac agggacatca aaagtaaaaa tgtgctgttg aaaaacaacc |
| | tgacagcttg cattgctgac tttgggttgg ccttaaaatt tgaggctggc |
| | aagtctgcag gcgataccca tggacaggtt ggtacccgga ggtacatggc |
| | tccagaggta ttagagggtg ctataaactt ccaaagggat gcatttttga |
| | ggatagatat gtatgccatg ggattagtcc tatgggaact ggcttctcgc |
| | tgtactgctg cagatggacc tgtagatgaa tacatgttgc catttgagga |
| | ggaaattggc cagcatccat ctcttgaaga catgcaggaa gttgttgtgc |
| | ataaaaaaaa gaggcctgtt ttaagagatt attggcagaa acatgctgga |
| | atggcaatgc tctgtgaaac cattgaagaa tgttgggatc acgacgcaga |
| | agccaggtta tcagctggat gtgtaggtga aagaattacc cagatgcaga |
| | gactaacaaa tattattacc acagaggaca ttgtaacagt ggtcacaatg |
| | gtgacaaatg ttgactttcc toccaaagaa tctagtcta |
| 15 | atacttggta gatcagaaac tcaggagtgt cttttcttta |
| | atgctaattgggaaaaagac agaaccaatc aaactggtgt tgaaccgtgt |
| | tatggtgacaaagataaacg goggcattgt tttgctacct ggaagaatat |
| | ttctggttccattgaaatag tgaaacaagg ttgttggctg gatgatatca |
| | actgctatgacaggactgat tgtgtagaaa aaaaagacag ccctgaagta |
| | tatttttgttgctgtgaggg caatatgtgt aatgaaaagt tttcttattt |
| | tccggagatggaagtcacac agcccacttc aaatccagtt acacctaagc |
| | caccc |

1. A method of improving lung diffusion capacity in a patient with pulmonary arterial hypertension comprising administering sotatercept to the patient, wherein lung diffusion capacity in the patient is improved following administration of sotatercept.

2. The method of claim 1, wherein the lung diffusion capacity is lung diffusion capacity for carbon monoxide (DLCO).

3. The method of claim 2, wherein the diffusion capacity for carbon monoxide (DLCO) in a patient with pulmonary arterial hypertension is increased by at least 4%.

4. The method of claim 2, wherein the diffusion capacity for carbon monoxide (DLCO) in a patient with pulmonary arterial hypertension is increased by at least 8%.

5. A method of improving gas exchange in a patient with pulmonary arterial hypertension comprising administering sotatercept to the patient, wherein gas exchange is improved in the patient following administration of sotatercept.

6. A method of improving oxygenation in a patient with pulmonary arterial hypertension comprising administering sotatercept to the patient, wherein oxygenation in the patient is improved following administration of sotatercept.

7. The method of claim 6, wherein the oxygenation is arterial oxygen tension [PaO₂].

8. The method of claim 7, wherein the arterial oxygen tension [PaO₂] increased by at least 5 mmHg.

9. The method of claim 7, wherein the arterial oxygen tension [PaO₂] increased by at least 7 mmHg.

10. The method of claim 1, wherein sotatercept is administered to the patient at a dose from 0.3 mg/kg to 0.7 mg/kg.

11. The method of claim 1, wherein sotatercept is administered to the patient at a dose of 0.3 mg/kg.

12. The method of claim 1, wherein sotatercept is administered to the patient at a dose of 0.7 mg/kg.

13. The method of claim 1, wherein sotatercept is administered to the patient once every three weeks.

14. The method of claim 1, wherein sotatercept is administered intravenously.

15. The method of claim 1, wherein sotatercept is administered subcutaneously.


SEQUENCE LISTING (22 sequences)

SEQ ID NO: 1 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 2 - protein, 4 aa, synthetic construct
TGGG

SEQ ID NO: 3 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 344 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 343 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 5 aa, synthetic construct
TGGGG

SEQ ID NO: 7 - protein, 5 aa, synthetic construct
SGGGG

SEQ ID NO: 8 - protein, 5 aa, synthetic construct
GGGGS

SEQ ID NO: 9 - skipped

SEQ ID NO: 10 - protein, 4 aa, synthetic construct
GGGG

SEQ ID NO: 11 - protein, 4 aa, synthetic construct
SGGG

SEQ ID NO: 12 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 13 - protein, 100 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 14 - DNA, 1539 bp, Homo sapiens
  source 1..1539: mol_type = genomic DNA
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 15 - DNA, 345 bp, Homo sapiens
  source 1..345: mol_type = genomic DNA
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 16 - skipped

SEQ ID NO: 17 - protein, 4 aa, synthetic construct
GGGG

SEQ ID NO: 18 - protein, 5 aa, synthetic construct
TGGGG

SEQ ID NO: 19 - protein, 5 aa, synthetic construct
SGGGG

SEQ ID NO: 20 - protein, 4 aa, synthetic construct
TGGG

SEQ ID NO: 21 - protein, 4 aa, synthetic construct
SGGG

SEQ ID NO: 22 - protein, 5 aa, synthetic construct
GGGGS